(19) United States
(12) Patent Application Publication
(10) Pub. No.: US 2023/0055582 A1
(43) Pub. Date: Feb. 23, 2023

(54) COMPOSITIONS AND METHODS FOR TREATMENT OF INFLAMMATORY CONDITIONS AND DISEASES OF THE SKIN

(71) Applicants: Guy LANDVER, Ramat Hasharon (IL); Eitan MOOR, Jerusalem (IL)

(72) Inventors: Guy LANDVER, Ramat Hasharon (IL); Eitan MOOR, Jerusalem (IL)

(21) Appl. No.: 17/759,497

(22) PCT Filed: Jan. 25, 2021
(86) PCT No.: PCT/IL2021/050081

§ 371 Date: Jul. 26, 2022

(30) Foreign Application Priority Data
Jan. 26, 2020 (IL) 272253

(51) Int. Cl. A61K 31/05; A61K 36/185; A61K 31/192; A61K 31/085; A61K 31/352; A61K 31/353; A61K 36/47; A61K 31/164; A61K 9/00; A61P 29/00

(52) U.S. Cl. CPC A61K 31/05; A61K 36/185; A61K 31/192; A61K 31/085; A61K 31/352; A61K 31/353; A61K 36/47; A61K 31/164; A61K 9/0014; A61P 29/00

(57) ABSTRACT

The present invention provides compositions comprising an active agent combination consisting of at least two of a cannabinoid, e.g., CBD, Dragon's blood, and palmitoylethanolamide (PEA), which is useful for treatment of inter alia inflammatory diseases or conditions of the skin; and methods of use.

### TECHNICAL FIELD

The present invention provides compositions comprising a drug combination, more specifically a combination of at least two of a cannabinoid, Dragon's blood, and palmitoylethanolamide (PEA), which are useful for treatment of, e.g., inflammatory diseases or conditions of the skin, and methods of use.

### BACKGROUND ART

Cannabidiol (CBD) is a non-psychoactive phytocannabinoid known for its therapeutic potential for many diseases and syndromes, and especially its anti-inflammatory (Burstein, 2015) and anti-nociceptive (pain-reducing) effects (Aviram and Samuelly-Leichtag, 2017). The anti-inflammatory effects of CBD in skin and mucous membranes tissues have been established in several animal and cell models, and make CBD one of the most promising natural molecules for the treatment of diseases and conditions where inflammation of epithelial cells is involved (Sangiovanni et al., 2019; Sherrif et al., 2019). The exact mechanism of action of CBD in primary skin cells (keratinocytes) is yet to be clarified.

Keratinocytes express several cannabinoid receptors, mainly cannabinoid type 2 (CB2) and transient receptor potential (TRP) vanilloid type-1 (TRPV-1), which is also known as the capsaicin receptor and is involved in nociception and inflammation. However, CBD binds to both CB2 and TRPV-1 receptors with low affinity. The anti-inflammatory effect of CBD in keratinocytes is not fully antagonized by either CB2 or TRPV-1 antagonists, leading researchers to suggest that the anti-inflammatory effects of CBD acts via the endocannabinoid system in the skin, by elevating the concentrations of the major endocannabinoid anandamide, also known as N-arachidonoylethanolamine (Petrosino et al., 2018; Bisogno et al., 2001).

Despite the promising pre-clinical results and the fact that CBD is widely used in skin care products, systematic clinical research of CBD in skin diseases was lacking until recently. In April 2019, Palmieri et al. published a clinical study where CBD-enriched ointment was administered to patients with different inflammatory dermal symptoms. CBD improved the symptoms and had a positive effect on all the parameters measured such as skin elasticity, hydration levels and scar size.

Dragon's Blood (DB) is the common name for several deep red resins and saps from trees used in traditional medicine (Salatino et al., 2007, Gupta et al., 2008). One of the most well-known variety is the resin of the South American tree Croton lechleri known in traditional medicine as Sangre de Grado/Drago. It is used in folk medicine for hundreds of years and for a variety of ailments and injuries, but particularly for the treatment of wounds and intestinal disorders. In addition to proanthocyanidins, which form the bulk of the resin, it further contains significant amounts of polyphenolic compounds such as flavonoids, and the alkaloid Taspine to which the wound healing properties of DB are ascribed in part (Vaisberg et al., 1989; Pona et al., 2019). The resin has a strong antioxidant, as well as antibacterial, antiviral and antifungal activity. In addition, it has immunomodulatory action and acts as an anti-inflammatory agent (Risco et al., 2003). DB accelerates wound healing and tissue regeneration in rats (Pieters et al., 1995) and improves wound healing in clinical trials (Namjoyan et al., 2016).

Palmitoylethanolamide (PEA) is an endogenous fatty acid amide related to endocannabinoids. Besides the human body, it is also found in many plants and animal tissues, as well as in food, e.g., soybeans, egg yolk, and peanuts. PEA is not considered a classic endocannabinoid because it has no affinity to the cannabinoid receptors CB1 and CB2. However, it acts on several cannabinoid-like receptors and is involved in the entourage effects of endogenous anandamide. PEA has anti-inflammatory effects in vitro and in vivo which are mediated, at least in part, by Peroxisome Proliferator-activated receptors (PPARs) and mainly through activation of PPAR-a (Peritore et al., 2019; Petrosino and Di Marzo, 2017). Also, PEA binds with high affinity to the G protein-coupled receptor 55 (GPR55) orphan receptor, but the physiological and therapeutic significance of this action are unclear. Another mechanism by which PEA may affect pain and inflammation is through enhancing the effects of endocannabinoids. PEA inhibits the degradation of anandamide, the major endogenous cannabinoid, and therefore increases its binding to cannabinoid receptors (Bisogno et al., 2001). In addition, PEA activates the TRPV-1 receptor. PEA binds to TRPV-1 receptor with low affinity, so TRPV-1 activation by PEA is probably indirect (Bisogno et al., 2001; Muller et al., 2019).

The anti-inflammatory properties of PEA were demonstrated in both cells and animal models. PEA reduces inflammatory cytokines in human immortalized keratinocytes (HaCaT cells) model of contact allergic dermatitis (Petrosino et al., 2010). Adelmidrol, a semi-synthetic analogue of PEA, is shown to reduce inflammation in cutaneous allergic inflammatory response model in Beagle dogs (Cerrato et al., 2012). PEA is available in different topical and systemic formulations for the treatment of chronic pain and chronic inflammation (Keppel and Kopsky, 2013). It has also been shown to be more effective than ibuprofen in temporo-mandibular inflammatory joint pain (Marini et al., 2012).

SUMMARY OF INVENTION

Using a cellular model for skin inflammation based on polyinosinic:polycytidylic acid (poly I:C) in immortalized keratinocytes (HaCaT cells), it has now been surprisingly found that while each one of CBD, Dragon's blood and PEA reduces the major cytokines associated with inflammation in said model, i.e., colony-stimulating factor (GM-CSF), interleukin 6 (IL-6), IL-8, monocyte chemoattractant protein-1 (MCP-1) and tumor necrosis factor alpha (TNF-α); and exerts a protective effect on HaCaT cells as measured from the cell viability, administration of two of the active agents together reduces inflammatory cytokines even further than each one of the active agents alone, wherein the particular combination of CBD with Dragon's blood also enhances the protective effects of each of them alone, as shown by further increase in cell viability.

Immortalized keratinocytes (the epithelial cells of the upper skin), e.g., HaCaT cells, are commonly used as a model for skin inflammatory response (Colombo et al., 2017) and contain a functioning system of inflammatory cytokines and cannabinoids (Toth et al., 2019). Poly I:C is an immunostimulant often used as a model for viral infection in epithelial cells of the lung (Lever et al., 2015), skin (Castex-Rizzi et al., 2014) and other tissues, where either the level of inflammation-related cytokines or their mRNA are measured. The model used in this study is based on the studies carried out by Petrosino et al. (2010 and 2018), where poly I:C stimulation was used as a model for contact dermatitis.

In one aspect, the present invention thus provides a composition comprising, as active agents, a combination consisting of at least two of (i) a cannabinoid, e.g., CBD, or an enantiomer, diastereomer, or mixture thereof, or a Cannabis plant extract comprising said cannabinoid; (ii) Dragon's blood; and (iii) palmitoylethanolamide (PEA), hereinafter referred to as “the active agents”. Particular such compositions comprise, as the active agents, said cannabinoid or Cannabis plant extract, and Dragon's blood; said cannabinoid or Cannabis plant extract, and PEA; Dragon's blood, and PEA; or said cannabinoid or Cannabis plant extract; Dragon's blood; and PEA.

The composition of the present invention may be either a pharmaceutical composition or a cosmetic (cosmeceutical) composition, formulated for topical application to the skin or a mucous membrane of a subject, and is beneficial in treatment of an inflammatory disease or condition of the skin, or a painful symptom of the skin, a mucous membrane, or a superficial muscle; minimizing scar formation after injury or pregnancy; wound healing; or anti-aging remedy.

In another aspect, the present invention relates to at least two of (i) a cannabinoid, e.g., CBD, or an enantiomer, diastereomer, or mixture thereof, or a Cannabis plant extract comprising said cannabinoid; (ii) Dragon's blood; and (iii) PEA, for use as a combination in the treatment of an inflammatory disease or condition of the skin, or a painful symptom of the skin, a mucous membrane, or a superficial muscle; minimizing scar formation after injury or pregnancy; wound healing; or anti-aging remedy.

In a further aspect, the present invention relates to a method for treatment of a disease, disorder, or condition of the skin or mucous membrane where inflammation is a significant component of the pathological profile, e.g., an inflammatory disease or condition of the skin, or a painful symptom of the skin, a mucous membrane, or a superficial muscle; minimizing scar formation after injury or pregnancy; wound healing; or anti-aging remedy, in a subject in need thereof, said method comprising topically applying to the skin or said mucous membrane of said subject an effective amount of a combination of at least two of the active agents. According to the invention, the combination of two or three of the active agents may be applied to the skin or mucous membrane of said subject either from two or three separate compositions (each comprising a different one of the active agents), e.g., two or three pharmaceutical or cosmetic compositions, or from a sole composition. In a particular such aspect, the present invention thus relates to a method for treatment of an inflammatory disease or condition of the skin, or a painful symptom of the skin, a mucous membrane, or a superficial muscle; minimizing scar formation after injury or pregnancy; wound healing; or anti-aging remedy, in a subject in need thereof, comprising topically applying to the skin or said mucous membrane of said subject an effective amount of a composition as disclosed herein, i.e., a pharmaceutical or cosmetic composition comprising two or three of the active agents, as defined above.

In yet another aspect, the present invention relates to a composition comprising a combination consisting of at least two of the active agents, for use in the treatment of an inflammatory disease or condition of the skin, or a painful symptom of the skin, a mucous membrane, or a superficial muscle; minimizing scar formation after injury or pregnancy; wound healing; or anti-aging remedy.

### BRIEF DESCRIPTION OF DRAWINGS

FIGS. 1A-1B show cell viability (1A) and five inflammatory cytokines after treatment with three different concentrations of the inflammation-inducing compound poly I:C (1B). Viability is given as O.D. values, and the cytokines are all in pg/mL.

FIG. 2 shows the effects of chosen concentrations of PEA, DB and CBD on cell viability and the levels of five cytokines associated with inflammation. All results are shown as percent of those measured after treatment with the inflammation-inducing poly I:C (100 μg/mL) alone.

FIG. 3 shows the effects of chosen concentrations of PEA, DB and CBD and some of their combinations on cell viability and the levels of five cytokines associated with inflammation. All results are shown as percent of those measured after treatment with the inflammation-inducing poly I:C (100 μg/mL) alone.

### DETAILED DESCRIPTION

In one aspect, the present invention provides a composition comprising, as active agents, a combination consisting of at least two of (i) a cannabinoid or a Cannabis plant extract comprising said cannabinoid; (ii) Dragon's blood; and (iii) PEA (“the active agents”).

The term “cannabinoid” as used herein refers to a chemical compound acting on cannabinoid receptors, i.e., a cannabinoid type 1 (CB1) or cannabinoid type 2 (CB2) receptor agonist. Ligands for these receptor proteins include the endocannabinoids produced naturally in the body; the phytocannabinoids found in Cannabis sativa and some other plants; and synthetic cannabinoids.

The cannabinoid used according to the present invention, in combination with one or two of the other active agents, i.e., one or two of said Dragon's blood and said PEA, may be derived from a Cannabis extract, e.g., Cannabis Sativa extract, using any suitable extraction and purification procedures known in the art. Alternatively, said cannabinoid may be synthesized following any one of the procedures disclosed in the literature.

In certain embodiments, the cannabinoid used in combination with one or two of the other active agents is selected from cannabidiol (CBD), cannabidiolic acid (CBDA), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-THCVA, Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, or pharmaceutically acceptable salt thereof, or a mixture thereof.

In particular embodiments, the cannabinoid used in combination with one or two of the other active agents is CBD (2-[(1R,6R)-6-isopropenyl-3-methylcyclohex-2-en-1-yl]-5-pentylbenzene-1,3-diol), or an enantiomer, diastereomer, or mixture (e.g., racemate) thereof, or a Cannabis plant extract, e.g., Cannabis Sativa extract, comprising CBD. More particular such compositions are those wherein the ratio between said CBD, or enantiomer, diastereomer, or mixture thereof and said Dragon's blood, when both present, is from about 1:0.15 to about 1:30 by weight, respectively; the ratio between said CBD, or enantiomer, diastereomer, or mixture thereof and said PEA, when both present, is from about 1:0.2 to about 1:20 by weight, respectively; and the ratio between said PEA and said Dragon's blood, when both present, is from about 1:0.1 to about 1:20 by weight, respectively.

CBD has two stereogenic centers, i.e., at positions 3 and 4 of the cyclohexenyl ring, and may accordingly exist as an enantiomer, i.e., an optical isomer (R or S, which may have an optical purity of 90%, 95%, 99% or more), racemate, i.e., an optically inactive mixture having equal amounts of R and S enantiomers, a diastereoisomer, or a mixture thereof. The present invention encompasses the use of all such enantiomers, isomers, and mixtures thereof.

CBD may be synthesized following any one of the procedures known in the art, e.g., by acid condensation of p-mentha-2,8-dien-1-ol with olivetol. Optically active forms of CBD may be prepared using any one of the methods disclosed in the art, e.g., by resolution of the racemic form by recrystallization techniques; chiral synthesis; extraction with chiral solvents; or chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

In particular embodiments, the composition of the invention comprises a Cannabis plant extract comprising a cannabinoid as referred to hereinabove, e.g., CBD. Such an extract may be obtained utilizing any method or technique known in the art, or may alternatively be a commercially available product. In specific such embodiments, the Cannabis plant extract is obtainable from the seeds of said Cannabis plant such as hemp oil (hempseed oil).

Dragon's blood is a bright red hydro-glycolic extract obtained from different species of several plant genera: Croton, Dracaena, Daemonorops, Calamus, and Pterocarpus. The resin has been in continuous use since ancient times as varnish, incense, and dye, as well as in folk medicine, In folk medicine, Dragon's blood is used externally, as self-forming latex bandage or as a wash to promote healing of wounds and to stop bleeding, and internally through ingestion for chest pains, post-partum bleeding, internal traumas and menstrual irregularities.

In certain embodiments, the Dragon's blood used in combination with one or two of the other active agents, i.e., one or two of said cannabinoid and said PEA, is obtainable from a plant selected from a Croton species, e.g., Croton lechleri, Croton palanostigma, Croton draconoides, and Croton urucurana; Dracaena species, e.g., Dracaena cinnabai, Dracaena draco, and Dracaena cochinchinensis; Daemonorops species, e.g., Daemonorops draco and Daemonorops rohtang; Calamus species, e.g., Calamus rotang; or Pterocarpus species, e.g., Pterocarpus indicus and Pterocarpus soyauxii. In particular embodiments, the Dragon's blood used in combination with one or two of the other active agents is obtainable from the tree Croton lechleri, in particular from the latex of said tree.

The Dragon's blood used according to the present invention, in combination with one or two of the other active agents, may be obtainable from any one of the plant species listed above using any suitable method or technique, e.g., by cutting the tree bark and collecting the sap, like in natural rubber. The natural latex resin obtained may be used as unmodified resin. Alternatively, the natural resin may be dried and re-dissolved in a suitable solvent, e.g., ethanol, methanol, petroleum ether, or chloromethane. These solutions can be either concentrated for use or filtered, vacuum evaporated and dissolved again (in this respect, see Gupta et al., 2011). Alternatively, the Dragon's blood used may be any one of the liquid- or powder-based Dragon's blood products commercially available, e.g., the liquid Dragon's blood obtained from Croton lechleri and marketed by Byron Bay (Australia).

In certain embodiments, the composition of the present invention according to any one of the embodiments defined above comprise, as the active agents, said cannabinoid or Cannabis plant extract; and said Dragon's blood. In particular such embodiments, said cannabinoid is CBD, or an enantiomer, diastereomer, or mixture (e.g., racemate) thereof; and the ratio between said CBD, or enantiomer, diastereomer, or mixture thereof and said Dragon's blood is from about 1:0.15 to about 1:30 (e.g., about 1:0.15 to about 1:25, about 1:0.15 to about 1:20, about 1:0.15 to about 1:15, or about 1:0.15 to about 1:10) by weight, respectively (hereinafter referred to as “a CBD-Dragon's blood composition”). More particular such compositions are those wherein the concentration of said cannabinoid in said composition is from about 3 mg/mL to about 30 mg/mL, e.g., about 4 mg/mL to about 26 mg/mL, about 6 mg/mL to about 22 mg/mL, about 8 mg/mL to about 18 mg/mL, or about 10 mg/mL to about 15 mg/mL; and the concentration of said Dragon's blood in said composition is from about 5 mg/mL to about 100 mg/mL, e.g., about 10 mg/mL to about 90 mg/mL, about 20 mg/mL to about 80 mg/mL, about 30 mg/mL to about 70 mg/mL, about 40 mg/mL to about 60 mg/mL, or about 50 mg/mL.

In certain embodiments, the composition of the present invention according to any one of the embodiments defined above comprise, as the active agents, said cannabinoid or Cannabis plant extract; and said PEA. In particular such embodiments, said cannabinoid is CBD, or an enantiomer, diastereomer, or mixture (e.g., racemate) thereof; and the ratio between said CBD, or enantiomer, diastereomer, or mixture thereof and said PEA is from about 1:0.2 to about 1:20 (e.g., about 1:0.2 to about 1:16, about 1:0.2 to about 1:14, about 1:0.2 to about 1:12, or about 1:0.2 to about 1:10) by weight, respectively (hereinafter referred to as “a CBD-PEA composition”). More particular such compositions are those wherein the concentration of said cannabinoid in said composition is from about 3 mg/mL to about 30 mg/mL, e.g., about 4 mg/mL to about 26 mg/mL, about 6 mg/mL to about 22 mg/mL, about 8 mg/mL to about 18 mg/mL, or about 10 mg/mL to about 15 mg/mL; and the concentration of said PEA in said composition is from about 5 mg/mL to about 50 mg/mL, e.g., about 10 mg/mL to about 40 mg/mL, about 15 mg/mL to about 45 mg/mL, about 20 mg/mL to about 40 mg/mL, or about 25 mg/mL to about 35 mg/mL, or about 30 mg/mL.

In certain embodiments, the composition of the present invention according to any one of the embodiments defined above comprise, as the active agents, said Dragon's blood; and said PEA; and the ratio between said PEA and said Dragon's blood is from about 1:0.1 to about 1:20 (e.g., about 1:0.1 to about 1:18, about 1:0.1 to about 1:16, about 1:0.1 to about 1:14, about 1:0.1 to about 1:12, or about 1:0.1 to about 1:10) by weight, respectively (hereinafter referred to as “a Dragon's blood-PEA composition”). More particular such compositions are those wherein the concentration of said Dragon's blood in said composition is from about 5 mg/mL to about 100 mg/mL, e.g., about 10 mg/mL to about 90 mg/mL, about 20 mg/mL to about 80 mg/mL, about 30 mg/mL to about 70 mg/mL, about 40 mg/mL to about 60 mg/mL, or about 50 mg/mL; and the concentration of said PEA in said composition is from about 5 mg/mL to about 50 mg/mL, e.g., about 10 mg/mL to about 40 mg/mL, about 15 mg/mL to about 45 mg/mL, about 20 mg/mL to about 40 mg/mL, or about 25 mg/mL to about 35 mg/mL, or about 30 mg/mL.

In certain embodiments, the composition of the present invention according to any one of the embodiments above comprise, as the active agents, said cannabinoid or Cannabis plant extract; said Dragon's blood; and said PEA. In particular such compositions, said cannabinoid is CBD, or an enantiomer, diastereomer, or mixture (e.g., racemate) thereof; the ratio between said CBD, or enantiomer, diastereomer, or mixture thereof and said Dragon's blood is from about 1:0.15 to about 1:30 (e.g., about 1:0.15 to about 1:25, about 1:0.15 to about 1:20, about 1:0.15 to about 1:15, or about 1:0.15 to about 1:10) by weight, respectively; the ratio between said CBD, or enantiomer, diastereomer, or mixture thereof and said PEA is from about 1:0.2 to about 1:20 (e.g., about 1:0.2 to about 1:16, about 1:0.2 to about 1:14, about 1:0.2 to about 1:12, or about 1:0.2 to about 1:10) by weight, respectively; and the ratio between said PEA and said Dragon's blood is from about 1:0.1 to about 1:20 (e.g., about 1:0.1 to about 1:18, about 1:0.1 to about 1:16, about 1:0.1 to about 1:14, about 1:0.1 to about 1:12, or about 1:0.1 to about 1:10) by weight, respectively (hereinafter referred to as “a CBD-Dragon's blood-PEA composition”). More particular such compositions are those wherein the concentration of said cannabinoid in said composition is from about 3 mg/mL to about 30 mg/mL, e.g., about 4 mg/mL to about 26 mg/mL, about 6 mg/mL to about 22 mg/mL, about 8 mg/mL to about 18 mg/mL, or about 10 mg/mL to about 15 mg/mL; the concentration of said Dragon's blood in said composition is from about 5 mg/mL to about 100 mg/mL, e.g., about 10 mg/mL to about 90 mg/mL, about 20 mg/mL to about 80 mg/mL, about 30 mg/mL to about 70 mg/mL, about 40 mg/mL to about 60 mg/mL, or about 50 mg/mL; and the concentration of said PEA in said composition is from about 5 mg/mL to about 50 mg/mL, e.g., about 10 mg/mL to about 40 mg/mL, about 15 mg/mL to about 45 mg/mL, about 20 mg/mL to about 40 mg/mL, or about 25 mg/mL to about 35 mg/mL, or about 30 mg/mL.

The composition of the present invention according to any one of the embodiments defined above may comprise, in addition to the two or three active agents, one or more ingredients referred to herein as non-active ingredients such as antioxidants, botanical extracts, oils, odorants, preservatives, thickening agents, emollient agents, emulsifying agents, chelating agents, moisturizing agents, penetrating agents, or cooling agents.

Examples of antioxidants include, without being limited to, vitamin C, vitamin E, alpha lipoic acid, vitamin A/retinol, co-enzyme Q10, and rosemary extract.

Non-limiting examples of botanical extracts include a cucumber extract, green tea extract, chamomile extract, bamboo extract, and aloe vera extract.

Examples of oils include, without limiting, hemp oil, neem oil, argan oil, marula oil, rose-hip oil, helichrysum oil, and grape seed oil.

Examples of odorants include, without being limited to, an essential oil, and a natural fragrance.

Non-limiting examples of preservatives include phenoxyethanol, Liquid Germal Plus, Naticide, Leucidal Liquid, potassium sorbate, and benzoic acid.

Examples of thickening agents include, without limiting, xantham gum, cellulose gum, Cera Bellina, and Emulsigel.

Examples of emollient agents include, without being limited to, a triglyceride, lanolin, lecithin, or shea butter.

Non-limiting examples of emulsifying agents include glyceryl stearate, Olivem 1000, a polysorbate such as polysorbate 20 and polysorbate 80, BTMS, and Polawax.

Examples of chelating agents include, without limiting, tetrasodium ethylenediaminetetraacetic acid (EDTA).

Examples of moisturizing agents include, without being limited to, glycerin, allantoin, panthenol, and isopropyl myristate.

Non-limiting examples of penetrating agents include propylene glycol, mannitol, liposomes, sodium PCA (the sodium salt of pyroglutamic acid) and squalene.

Examples of cooling agents include, without being limited to, menthol, N-Ethyl-2-isopropyl-5-methylcyclohexanecarboxamide (WS-3), and Frescolat ML.

In certain embodiments, the composition of the invention as defined in any one of the embodiments above is a pharmaceutical composition comprising a pharmaceutically acceptable carrier; or a cosmetic composition comprising a cosmetically acceptable carrier, formulated for topical application to the skin or to a mucous membrane of a subject. Such pharmaceutical and cosmetic compositions are beneficial in reduction/inhibition of the inflammatory process, regeneration of healthy skin, and protection against degradation and potential contamination, and are thus useful in treatment of a disease, disorder, or condition of the skin or mucous membrane where inflammation is a significant component of the pathological profile, e.g., an inflammatory disease or condition of the skin, or a painful symptom of the skin, a mucous membrane, or a superficial muscle; as well as in minimizing scar formation after injury or pregnancy; wound healing; or anti-aging remedy.

The pharmaceutical composition disclosed herein may be formulated, e.g., as a solution, emulsion, suspension, lotion, gel, cream, foam, ointment, or paste. In addition, such a composition may be formulated for transdermal administration as either a part of a patch or a coating layer on a medical device aimed at making contact with the skin and/or a mucous membrane of a subject.

Pharmaceutical compositions according to the present invention may be prepared by conventional techniques, e.g., as described in Remington: The Science and Practice of Pharmacy, 19^{th} Ed., 1995. The compositions can be prepared, e.g., by uniformly and intimately bringing the active agents into association with a liquid carrier, a finely divided solid carrier, or both, and then, if necessary, shaping the product into the desired formulation. The composition may be in the form of a liquid (e.g., solution, emulsion, suspension, or lotion), gel, or semisolid (e.g., cream, foam, ointment, paste), and may further include pharmaceutically acceptable fillers, carriers, diluents or adjuvants, and other inert ingredients and excipients.

The cosmetic composition disclosed herein may be formulated, e.g., as a solution, emulsion, suspension, lotion, gel, cream, paste, powder, soap, surfactant-containing cleanser, oil, powder foundation, emulsion foundation, wax foundation, and spray. For example, such a composition may be prepared in a dosage form of an emollient lotion, nourishing lotion, nourishing cream, massage cream, essence, eye cream, cleansing cream, cleansing foam, cleansing water, pack, spray, and/or powder. The cosmetic compositions may include, as carriers, ingredients ordinarily used in cosmetic compositions, e.g., ordinary supplements such as antioxidants, purifiers, solubilizers, vitamins, pigments and/or flavoring agents.

In cases the cosmetic composition is formulated as a solution or emulsion, the carrier ingredients may include, e.g., a solvent, solubilizer, or emulsifier such as water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, and fatty acid esters of sorbitan.

In cases the cosmetic composition is formulated as a suspension, the carrier ingredients may include, e.g., a liquid diluent such as water, ethanol, and propylene glycol; a suspending agent such as ethoxylated isostearyl alcohol, polyoxyethylene sorbitol ester, and polyoxyethylene sorbitan ester; microcrystalline cellulose, aluminum metahydroxide, bentonite, agar, or tragacanth.

In cases the cosmetic composition is formulated as a gel, cream or paste, the carrier ingredients may include, e.g., an animal oil, plant oil, wax, paraffin, starch, tragacanth, a cellulose derivative, polyethylene glycol, silicone, bentonite, silica, talc, or zinc oxide.

In cases the cosmetic composition is formulated as a spray or powder, the carrier ingredients may include, e.g., lactose, talc, silica, aluminum hydroxide, calcium silicate, or a polyamide powder. Cosmetic compositions formulated as a spray may further include a propellant such as chlorofluorohydrocarbon, propane/butane, and/or dimethyl ether.

Cosmetic compositions formulated as a surfactant-containing cleanser may include, as the carrier ingredient, an aliphatic alcohol sulfate, aliphatic alcohol ether sulfate, sulfosuccinate monoester, isethionate, imidazolium derivatives, methyl taurate, sarcosinate, fatty acid amide ether sulfate, alkyl amido betaine, aliphatic alcohol, fatty acid glyceride, fatty acid diethanolamide, plant oil, lanoline derivatives, or ethoxylated glycerol fatty acid ester.

As with pharmaceutical compositions, the amount of active agent(s) in the cosmetic composition will depend on several factors, but will generally comprise a concentration that is sufficient to provide a consumer with an effective amount of the active agent combination upon consumption of a regular (e.g., daily) portion of the composition.

In another aspect, the present invention relates to a combination of at least two of the three active agents, i.e., a cannabinoid or a Cannabis plant extract comprising said cannabinoid; Dragon's blood; and PEA, for use in the treatment of a disease, disorder, or condition of the skin or mucous membrane where inflammation is a significant component of the pathological profile, e.g., an inflammatory disease or condition of the skin, or a painful symptom of the skin, a mucous membrane, or a superficial muscle; minimizing scar formation after injury or pregnancy; wound healing; or anti-aging remedy. In a particular such aspect, said cannabinoid is CBD, or an enantiomer, diastereomer, or mixture (e.g., racemate) thereof; the ratio between said cannabinoid and said Dragon's blood, when both present, is from about 1:0.15 to about 1:30 by weight, respectively; the ratio between said cannabinoid and said PEA, when both present, is from about 1:0.2 to about 1:20 by weight, respectively; and the ratio between said PEA and said Dragon's blood, when both present, is from about 1:0.1 to about 1:20 by weight, respectively.

In certain embodiments, the active agent combination disclosed hereinabove consists of said cannabinoid or Cannabis plant extract; and said Dragon's blood. Particular such combinations are those wherein said cannabinoid is CBD, or an enantiomer, diastereomer, or mixture (e.g., racemate) thereof; and the ratio between said cannabinoid and said Dragon's blood is from about 1:0.15 to about 1:30 (e.g., about 1:0.15 to about 1:25, about 1:0.15 to about 1:20, about 1:0.15 to about 1:15, or about 1:0.15 to about 1:10) by weight, respectively (hereinafter referred to as “CBD-Dragon's blood combinations”).

In certain embodiments, the active agent combination disclosed hereinabove consists of said cannabinoid or Cannabis plant extract; and said PEA. Particular such combinations are those wherein said cannabinoid is CBD, or an enantiomer, diastereomer, or mixture (e.g., racemate) thereof; and the ratio between said cannabinoid and said PEA is from about 1:0.2 to about 1:20 (e.g., about 1:0.2 to about 1:16, about 1:0.2 to about 1:14, about 1:0.2 to about 1:12, or about 1:0.2 to about 1:10) by weight, respectively (hereinafter referred to as “CBD-PEA combinations”).

In certain embodiments, the active agent combination disclosed hereinabove consists of said Dragon's blood; and said PEA; and the ratio between said PEA and said Dragon's blood is from about 1:0.1 to about 1:20 (e.g., about 1:0.1 to about 1:18, about 1:0.1 to about 1:16, about 1:0.1 to about 1:14, about 1:0.1 to about 1:12, or about 1:0.1 to about 1:10) by weight, respectively (hereinafter referred to as “Dragon's blood-PEA combinations”).

In certain embodiments, the active agent combination disclosed hereinabove consists of said cannabinoid or Cannabis plant extract; said Dragon's blood; and said PEA. Particular such combinations are those wherein said cannabinoid is CBD, or an enantiomer, diastereomer, or mixture (e.g., racemate) thereof; the ratio between said cannabinoid and said Dragon's blood is from about 1:0.15 to about 1:30 (e.g., about 1:0.15 to about 1:25, about 1:0.15 to about 1:20, about 1:0.15 to about 1:15, or about 1:0.15 to about 1:10) by weight, respectively; the ratio between said cannabinoid and said PEA is from about 1:0.2 to about 1:20 (e.g., about 1:0.2 to about 1:16, about 1:0.2 to about 1:14, about 1:0.2 to about 1:12, or about 1:0.2 to about 1:10) by weight, respectively; and the ratio between said PEA and said Dragon's blood is from about 1:0.1 to about 1:20 (e.g., about 1:0.1 to about 1:18, about 1:0.1 to about 1:16, about 1:0.1 to about 1:14, about 1:0.1 to about 1:12, or about 1:0.1 to about 1:10) by weight, respectively (hereinafter referred to as “CBD-Dragon's blood-PEA combinations”).

In a further aspect, the present invention relates to a method for treatment of a disease, disorder, or condition of the skin or mucous membrane where inflammation is a significant component of the pathological profile, e.g., an inflammatory disease or condition of the skin, or a painful symptom of the skin, a mucous membrane, or a superficial muscle; minimizing scar formation after injury or pregnancy; wound healing; or anti-aging remedy, in a subject in need thereof, said method comprising topically applying to the skin or said mucous membrane of said subject an effective amount of a combination of at least two of (i) a cannabinoid or a Cannabis plant extract comprising said cannabinoid; (ii) Dragon's blood; and (iii) PEA. In certain embodiments, said cannabinoid is CBD, or an enantiomer, or diastereomer, or a mixture (e.g., racemate) thereof, and the combination thus applied is a CBD-Dragon's blood combination, a CBD-PEA combination, or a CBD-Dragon's blood-PEA combination. In other embodiments, the combination applied is a Dragon's blood-PEA combination. According to the method of the invention, the active agent combination may be topically applied to the skin or said mucous membrane of said subject either from two or three separate compositions, e.g., two or three pharmaceutical or cosmetic compositions, each containing a different one of the active agents, either concurrently or sequentially at any order, or from a sole composition.

In a particular such aspect, the present invention thus relates to a method for treatment of an inflammatory disease or condition of the skin, or a painful symptom of the skin, a mucous membrane, or a superficial muscle; minimizing scar formation after injury or pregnancy; wound healing; or anti-aging remedy, in a subject in need thereof, comprising topically applying to the skin or said mucous membrane of said subject an effective amount of a composition as disclosed herein, e.g., a CBD-Dragon's blood composition, a CBD-PEA composition, a Dragon's blood-PEA composition, or a CBD-Dragon's blood-PEA composition, as defined in any one of the embodiments above.

The term “subject” as used herein refers to any mammal, e.g., a human, non-human primate, horse, ferret, dog, cat, cow, and goat. In a preferred embodiment, the term “subject” denotes a human, i.e., an individual.

The term “mucous membrane” (also referred to as “mucosa”) as used herein denotes a membrane consisting of one or more layers of epithelial cells overlying a layer of loose connective tissue, which lines various cavities in the body and covers the surface of internal organs. Mucosa is mostly of endodermal origin and is continuous with the skin at various body openings such as the eyes, ears, inside the nose, inside the mouth, lip, vagina, the urethral opening and the anus. Some mucous membranes secrete mucus, a thick protective fluid. The function of the membrane is to stop pathogens and dirt from entering the body and to prevent bodily tissues from becoming dehydrated.

The term “treatment” as used herein generally refers to the applying of a an amount of an active agent combination as referred to in any one of the aspects of the present invention (e.g., a CBD-Dragon's blood combination, CBD-PEA combination, Dragon's blood-PEA combination, or CBD-Dragon's blood-PEA combination), which is effective to ameliorate undesired symptoms associated with the medical condition treated; prevent the manifestation of such symptoms before they occur; slow down the progression of said medical condition; slow down the deterioration of symptoms; enhance the onset of remission period; slow down the irreversible damage caused in the progressive chronic stage of said medical condition; delay the onset of said progressive stage; lessen the severity or cure said medical condition; improve survival rate or promote a more rapid recovery; and/or prevent said medical condition form occurring. According to the method of the present invention, the active agent combination as defined in any one of the embodiments herein may be applied to the skin or mucous membrane of the subject treated one or more times a day, e.g., once, twice, or three times a day; and over a time period of, e.g., days, weeks, months, years, or more.

The term “effective amount” as used herein means an amount of said active agent combination that will elicit the biological or medical response of a tissue, system, animal or human that is being sought. The amount must be effective to achieve the desired therapeutic effect as described above, depending inter alia on the type and severity of the condition to be treated and the treatment regime. The effective amount is typically determined in appropriately designed clinical trials (dose range studies) and the person versed in the art will know how to properly conduct such trials to determine the effective amount.

The term “treatment of an inflammatory disease or condition of the skin” as used herein refers to ameliorating undesired symptoms associated with said disease or condition; preventing the manifestation of such symptoms before they occur; slowing down the progression of said disease or condition; slowing down the deterioration of symptoms; enhancing the onset of remission period; slowing down the irreversible damage caused in the progressive chronic stage of said disease or condition; delaying the onset of said progressive stage; lessening the severity of, or curing said disease or condition; improving survival rate or promoting a more rapid recovery; and/or preventing said disease or condition form occurring.

Examples of inflammatory diseases or conditions of the skin treatable by the method of the invention include, without limiting, dermatitis including atopic and contact dermatitis, eczema, acne, psoriasis, piles, itching, Diaper rash, Nipple cracks, and burns.

The term “treatment of a painful symptom of the skin, a mucous membrane, or a superficial muscle” as used herein refers to ameliorating, i.e., lessening the severity of, or slowing down the deterioration of said painful symptom; and/or preventing the manifestation of said symptom before it occurs.

Examples of painful symptom of the skin, mucous membrane, or superficial muscle that can be treated by the method of the invention include, without being limited to, piles, menstrual cramps, ulcers of the mouth and digestive tract, burns, wounds and lacerations, cracks, and painful irritations caused by contacts with chemicals from synthetic, plant or animal sources. Additional examples include spasm of the vaginal muscles (in this case, the method of the invention may be aimed at minimizing genital soreness or pain during intercourse).

The term “minimizing scar formation after injury or pregnancy” as used herein means reducing the extent of scar-formation, or reducing the visibility, the surface, and/or the volume of scar tissue resulting from the healing process of an injury or excessive stretching.

The term “wound healing” as used herein refers to any one of the phases of a skin restoration following an injury, i.e., the inflammatory phase, the proliferation phase, and the maturation/remodeling phase. Improving the process of wound healing means minimizing secondary tissue damage cause by the inflammatory process; accelerating the re-epithelialization, contraction and closure of the wound; and preventing the formation of chronic injury and ulcers.

The term “anti-aging remedy” as used herein refers to a treatment aimed at reducing or inhibiting the formation of wrinkles and the appearance of different types of discolorations and stains associated with normal aging.

In yet another aspect, the present invention relates to a composition comprising a combination comprising at least two of the active agents according to any one of the embodiments above, e.g., a CBD-Dragon's blood combination, a CBD-PEA combination, Dragon's blood-PEA combination, or CBD-Dragon's blood-PEA combination, for use in the treatment of a disease, disorder, or condition of the skin or mucous membrane where inflammation is a significant component of the pathological profile, e.g., an inflammatory disease or condition of the skin, or a painful symptom of the skin, a mucous membrane, or a superficial muscle; as well as in minimizing scar formation after injury or pregnancy; wound healing; or anti-aging remedy.

As well-known to any person skilled in the art, in a multi drug-based treatment, both the exact ratio between the drugs administered as well as the timing, dosing and pharmacokinetic aspects, play an extremely important role. In other words, in order to determine the optimal ratio between the active agents topically applied, not only the combined/synergistic efficacy and potency are of importance, but also the relative pharmacokinetics of each drug and the optimal formulation. Thus, in certain embodiments, the weight ratio of the active agents applied is precisely calibrated and said active agents are preferably formulated for optimal pharmacokinetic performance and efficacy and for subject's compliance. Moreover, the synergistic effects between the active agents in the active agent combination may depend on the time they have to act together in the body, i.e., on the relative release profile of the drugs determined by the formulation of each one of the drugs in the combination.

Unless otherwise indicated, all numbers expressing, e.g., ratios between active agents and concentrations thereof in the compositions disclosed herein, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Materials and Methods

Cell culture. The human keratinocyte cell line HaCaT was a kind gift from Prof. Joseph Kost (Department of Chemical Engineering, Ben-Gurion University of the Negev, Israel). Cells were cultured in Dulbecco's Modified Eagle Medium (DMEM) (Biological Industries, Kibbutz Beit-Haemek, Israel) supplemented with 1% antibiotic-antimycotic (Tivan Biotech, Kfar-Saba, Israel), 1mM sodium pyruvate, and 10% fetal bovine serum (FBS) (Biological Industries), and were maintained in an incubator with 5% CO₂ at 37° C.

CBD, PEA and DB. CBD was purchased from Sigma Israel. DB was purchased from Byron Bay (Australia), and PEA was purchased from JP Russel science global Ltd. (Netherlands). CBD was 3.18 mM solution in ethanol. DB was in pure liquid form. PEA was in powder form. A 30 mM stock solution was made by dissolving PEA in dimethyl sulfoxide (DMSO). DB was diluted directly in the cell medium to maximum of 0.1%. All stocks were filtered using a 0.2 μm filter before use and further diluted to the required concentration in cell medium. Vehicle (control) solutions contained the corresponding amount of DMSO (0.007%).

Procedure and administration of active compounds. 90,000 cells/well were plated on 12-well plates and allowed to attach at 37° C. for 24 hours. Subsequently, the wells were washed with phosphate-buffered saline (PBS) and the medium was replaced with a medium containing either vehicle (control) or poly I:C. CBD, PEA and/or DB were administered in the medium together with poly I:C. The wells were then incubated for further 24 h and 50μL samples were drawn for analysis of cytokines. Samples were frozen and shipped on dry ice.

Cell viability assay. Cell viability was measured using a crystal violet assay. 90,000 cells/well were plated on 12-well plates and allowed to attach at 37° C. for 24 hours. Cells were then treated with poly I:C alone or in combination with CBD, PEA and/or DB. 24 hours later medium was aspirated, and cells were washed twice with PBS. 500 μL of crystal violet staining solution (0.5% (w/v) crystal violet powder, 20% methanol) was added to each well, followed by incubation for 30 min at room temperature. The staining solution was discarded, the plates were washed 5 times with distilled water and dried, and 500 μL of 10% acetic acid (v/v) was added to each well. Absorbance was measured at 595 nm.

Analysis of cytokine content. Samples were shipped to Eve Technologies, Calgary (Canada) for analysis. Samples were analyzed using Human Focused 13-Plex Discovery Assay, a Multiplex immunoassay with fluorometric detection. Preliminary tests were conducted in order to determine samples dilution. The cytokines measured were granulocyte-macrophage colony-stimulating factor (GM-CSF), interferon gamma (IFNy), interleukin 1 beta (IL-1B), IL-2, IL-4, IL-5, IL-6, IL-8, IL-10, IL-12, IL-13, monocyte chemoattractant protein-1 (MCP-1) and tumor necrosis factor alpha (TNF-α). Results were calculated only for GM-CSF, IL-6, IL-8, MCP-1 and TNF-α.

Toxicity. HaCaT cells were incubated in 12 wells plates until 60-70% confluency was achieved. Subsequently, the cells were washed and incubated with medium containing PEA, DB or CBD. After 50 hours of incubation, the wells were observed under a light microscope and toxicity of the active compounds was assessed qualitatively by comparing the treated cells to controls incubated with vehicle. The results are presented in the Table 1. As concluded, under the conditions tested, PEA can be safely used in concentration of up to 50 μM, DB is non-toxic at concentrations of up to 0.01%, and CBD is non-toxic at concentration of up to 15 μM.

TABLE 1
Results of toxicity experiments on HaCat cells.

| Treatment | Concentration | | Toxicity |
|---|---|---|---|
| PEA | 30 | μM | non toxic |
| | 50 | μM | non toxic |
| | 100 | μM | toxic |
| DB | 0.003% | | non toxic |
| | 0.01% | | non toxic |
| | 0.03% | | toxic |
| | 0.1% | | very toxic |
| CBD | 5 | μM | non toxic |
| | 15 | μM | non toxic |
| | 45 | μM | very toxic |

### Results

All the results shown were obtained from the same experiment and represent an average of 3 measurements from three different wells (triplicates). All measurement were within the linear portion of the standard curved, except those of baseline TNF-α, which were slightly lower than the lower end of the standard curve (0.64 pg/mL).

Different Concentrations of poly I:C

Viability (calculated from the optical density, O.D) and cytokine concentrations in the medium were measured after treatment with different concentrations of poly I:C. The concentrations used were 20, 50 and 100m/mL. Table 2 shows the values measured 24 h after treatment with 3 different concentration of poly I:C and control (vehicle), wherein each value represents an average of three measurements from 3 different wells in pg/mL. In TNF-α, control values were below the standard curve and therefore, may be under or over estimated. Table 3 shows the data shown in Table 2, presented as percent of control values.

TABLE 2

| | Cell | | | | | |
|---|---|---|---|---|---|---|
| | viability | GM-CSF | IL-6 | IL-8 | MCP-1 | TNF-α |
| Control | 2.371 | 19.903 | 2.687 | 198.707 | 52.42 | 0.323 |
| Poly I:C 20 μg/mL | 1.827 | 53.09 | 14.07 | 657.01 | 369.54 | 2.74 |
| Poly I:C 50 μg/mL | 1.714 | 62.68 | 19.50 | 729.48 | 711.76 | 3.81 |
| Poly I:C 100 μg/mL | 1.259 | 76.98 | 35.14 | 918.50 | 1727.32 | 6.47 |

TABLE 3

| | GM-CSF | IL-6 | IL-8 | MCP-1 | TNF-α |
|---|---|---|---|---|---|
| Poly I:C 20 μg/mL | 266.74 | 523.57 | 330.64 | 704.95 | 846.39 |
| Poly I:C 50 μg/mL | 314.94 | 725.81 | 367.11 | 1357.80 | 1179.38 |
| Poly I:C 100 μg/mL | 386.75 | 1307.82 | 462.24 | 3295.15 | 2001.03 |

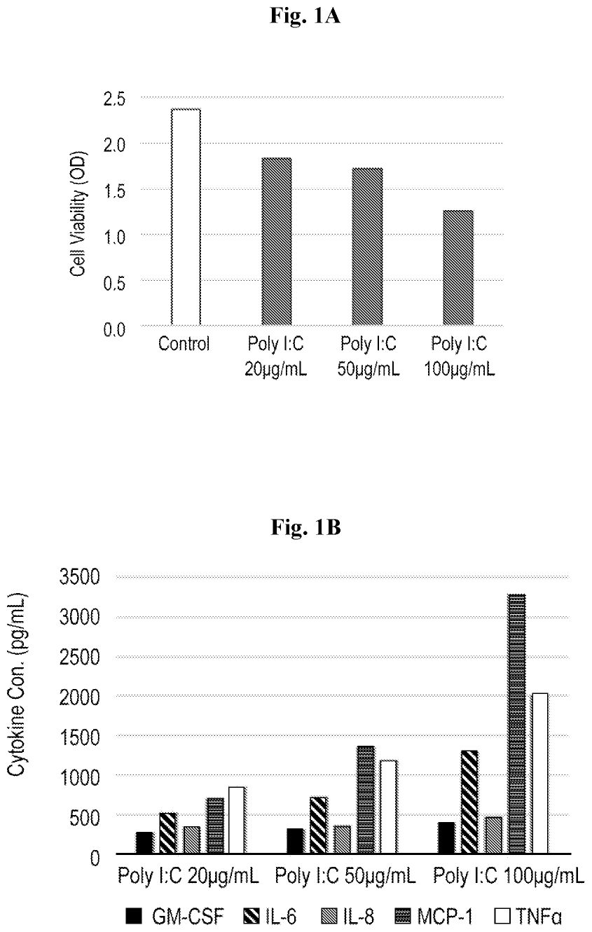

FIG. 1 shows cell viability (1A) and five inflammatory cytokines after treatment with three different concentrations of the inflammation-inducing compound poly I:C (1B). Viability is given as O.D. values, and the cytokines are all in pg/mL. In general, the results show dose-dependent decrease in cell viability with simultaneous increase in inflammatory cytokines. Viability was reduced by almost 50% at poly I:C 100 μg/mL. Pronounced increase in cytokine concentration was observed, up to 33-fold for MCP-1 at poly I:C 100 μg/mL. GM-CSF and IL-8 were less affected by poly I:C treatment than IL-6, MCP-1 and TNF-α.

The effects of CBD, PEA and Dragon Blood

CBD, PEA and DB were dissolved and added to the cell medium together with poly I:C 100 μg/mL. Cell viability and the concentrations of five cytokines related to inflammation were measured in samples taken from the medium of the cells. Table 4 shows the cell viability, as well as the concentrations of the five cytokines in the cell medium (pg/mL), and Table 5 shows the same data, presented as percent of values measured after treatment with poly I:C alone.

TABLE 4

| | Cell | | | | | |
|---|---|---|---|---|---|---|
| | viability | GM-CSF | IL-6 | IL-8 | MCP-1 | TNF-α |
| Control | 2.371 | 19.90 | 2.69 | 198.71 | 52.42 | 0.323 |
| Poly I:C 100 | 1.259 | 76.98 | 35.14 | 918.50 | 1727.32 | 6.470 |
| PEA 0.2 μM | 1.443 | 60.36 | 27.43 | 810.38 | 1345.28 | 4.673 |
| PEA 2 μM | 1.413 | 57.54 | 27.23 | 773.38 | 1554.20 | 4.470 |
| DB 0.003% | 1.830 | 61.79 | 26.26 | 826.32 | 1384.53 | 4.643 |
| DB 0.01% | 1.410 | 66.70 | 31.33 | 766.93 | 1394.52 | 5.383 |
| CBD 3 μM | 1.328 | 56.44 | 18.95 | 643.44 | 956.97 | 3.277 |
| CBD 10 μM | 1.451 | 49.55 | 12.15 | 335.44 | 272.60 | 1.517 |

TABLE 5

| | Cell | | | | | |
|---|---|---|---|---|---|---|
| | viability | GM-CSF | IL-6 | IL-8 | MCP-1 | TNF-α |
| Poly I:C 100 | 100.00 | 100.00 | 100.00 | 100.00 | 100.00 | 100.00 |
| PEA 0.2 μM | 114.66 | 78.42 | 78.08 | 88.23 | 77.88 | 72.23 |
| PEA 2 μM | 112.28 | 74.75 | 77.49 | 84.19 | 89.98 | 69.09 |
| DB 0.003% | 145.40 | 80.27 | 74.73 | 89.96 | 80.15 | 71.77 |
| DB 0.01% | 112.01 | 86.65 | 89.16 | 83.50 | 80.73 | 83.20 |
| CBD 3 μM | 105.50 | 73.32 | 53.94 | 70.05 | 54.77 | 50.64 |
| CBD 10 μM | 115.29 | 64.37 | 34.57 | 36.52 | 15.78 | 23.44 |

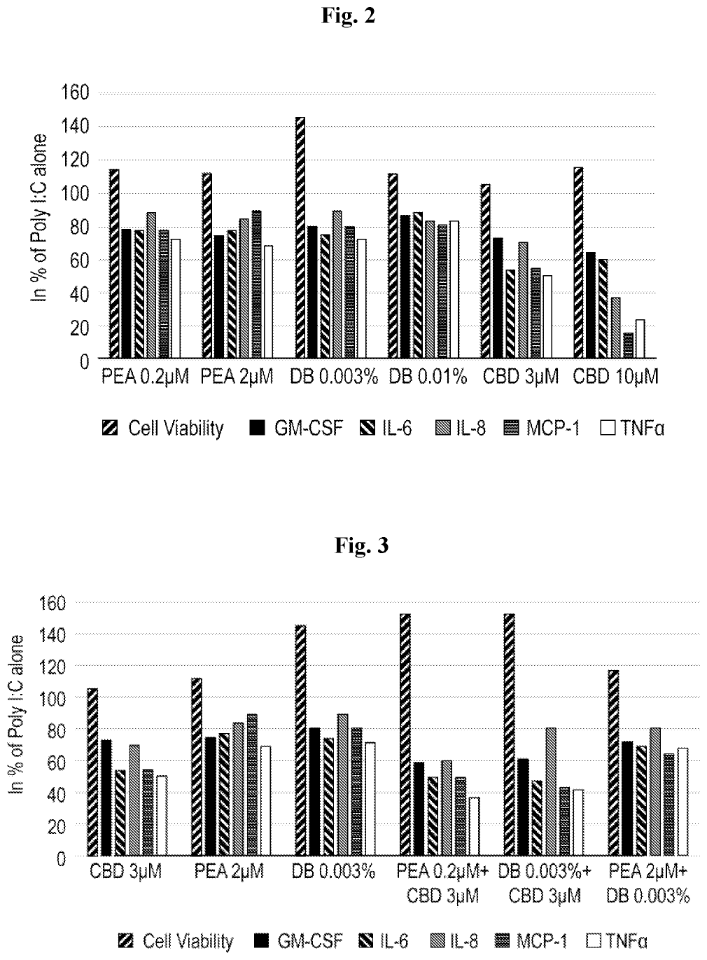

FIG. 2 shows the effects of chosen concentrations of PEA, DB and CBD on cell viability and the levels of five cytokines associated with inflammation. All results are shown as percent of those measured after treatment with the inflammation-inducing poly I:C (100 μg/mL) alone.

CBD, PEA and DB, each separately, reduced the levels of the five cytokines and increased the viability of the cells.

Cell viability increased by 4.5% to 45.4% by the active compounds; however, only the increase induced by CBD was concentration dependent (5.5% and 15.3% increase with 3 μM and 10 μM, respectively). PEA and DB increased cell viability significantly, but the effect of PEA was similar regardless the concentration used, and the effect of DB was lower at the higher concentration. DB at 0.003% had a particularly strong positive effect on cell viability (45.4% increase).

Cytokines were all reduced by the active compounds at all concentrations; however, not all cytokines measured were equally affected. GM-CSF was the least affected (13% to 36% decrease), while CMP-1 was reduced by up to 84%.

CBD was the most potent in reducing cytokine levels even at the lower concentration (3 μM) and this effect was even more pronounced with the higher concentration (10 μM). PEA reduced the concentrations of cytokines at 0.2 μM by 12% to 28%. With 2.0 μM PEA, a small further reduction of cytokine levels was observed for all cytokines but MCP-1. Treatment with 0.003% DB caused a reduction of cytokines in the range of 10-28%, similarly to PEA, but the higher concentration, 0.01% was less effective in reducing cytokine levels in all except IL-8.

In summary, all the active compound tested increased the cell viability of poly I:C treated cells and reduced the levels of cytokines as compared to poly I:C alone. DB had the strongest effect on cell viability, while CBD was highly effective in reducing cytokine levels and this effect was dose dependent. All cytokines measured were reduced by 60-80% by the higher dose of CBD.

### The Effects of Combinations CBD, PEA and Dragon Blood

Different combinations of CBD, PEA and DB were tested in poly I:C treated cells. As expected, these combinations also reduced the levels of inflammatory cytokines and increased cell viability. Table 6 shows cell viability (in O.D.) and cytokine concentrations (in pg/mL) following treatment with the inflammation inducing agent poly I:C (100 μg/mL), CBD, PEA, DB, or a combination of two of the active agents (CBD+PEA, CBD+DB and PEA+DB). Table 7 shows the same data, presented as percent of values measured after treatment with poly I:C alone (positive control).

TABLE 6

| | Cell | | | | | |
|---|---|---|---|---|---|---|
| | viability | GM-CSF | IL-6 | IL-8 | MCP-1 | TNF-α |
| CBD 3 μM | 1.328 | 56.44 | 18.95 | 643.44 | 945.97 | 3.277 |
| PEA 2 μM | 1.413 | 57.54 | 27.23 | 773.27 | 1554.20 | 4.470 |
| DB 0.003% | 1.830 | 61.79 | 26.26 | 826.32 | 1384.53 | 4.643 |
| PEA 0.2 μM + CBD 3 μM | 1.300 | 45.54 | 17.71 | 551.43 | 854.41 | 2.413 |
| DB 0.003% + CBD 3 μM | 1.918 | 46.77 | 16.66 | 737.43 | 750.77 | 2.710 |
| PEA 2 μM + DB 0.003% | 1.473 | 55.54 | 24.50 | 737.43 | 1116.64 | 4.393 |

TABLE 7

| | Cell | | | | | |
|---|---|---|---|---|---|---|
| | viability | GM-CSF | IL-6 | IL-8 | MCP-1 | TNF-α |
| CBD 3 μM | 105.50 | 73.32 | 53.04 | 70.05 | 54.77 | 50.64 |
| PEA 2 μM | 112.28 | 74.75 | 77.49 | 84.19 | 89.98 | 69.09 |
| DB 0.003% | 145.40 | 80.27 | 74.73 | 89.96 | 80.15 | 71.77 |
| PEA 0.2 μM + CBD 3 μM | 103.30 | 59.16 | 50.39 | 60.04 | 49.46 | 37.30 |
| DB 0.003% + CBD 3 μM | 152.37 | 60.76 | 47.43 | 80.29 | 43.46 | 41.89 |
| PEA 2 μM + DB 0.003% | 117.04 | 72.15 | 69.74 | 80.29 | 64.65 | 67.90 |

FIG. 3 shows the effects of chosen concentrations of PEA, DB and CBD and some of their combinations on cell viability and the levels of five cytokines associated with inflammation. All results are shown as percent of those measured after treatment with the inflammation-inducing poly I:C (100 μg/mL) alone.

The data presented above show that every combination of two out of the three anti-inflammatory agents reduces the inflammatory cytokines more effectively than each of said agents alone, at least in the concentration range tested. This finding is consistent for all five cytokines measured. The effects of these combinations on cell viability are slightly different. The combination of CBD and DB increases the cell viability further than either of them alone. However, while PEA alone increases cell viability, the increase caused by the combination of PEA and CBD is smaller, and even smaller than that caused by CBD alone. This increase in cell viability caused by the combination of PEA and DB was higher than that caused by PEA alone, but yet significantly lower than that caused by DB alone.

### Conclusions

The present study is largely based on a study published by Petrosino et al. (2018), where CBD was administered to HaCaT cells stimulated with poly I:C. Although the study focused on monocyte chemoattractant protein 2 (MCP2), an early response cytokine that stimulates mast cells, but also keratinocytes, they also measured IL-6, IL-8 and TNF-α. The present results are in good agreement with those of Petrosino et al.

The results of this study demonstrate that CBD, PEA and DB all reduce the major cytokines associated with inflammation in the present HaCaT cell/poly I:C model of inflammation, and that all three exert a protective effect on HaCaT cells as measured from the cell viability. The results further demonstrate that administration of two of the active compounds together reduces inflammatory cytokines even further than each active alone, and the combination of CBD with DB also enhances the protective effects of each of them alone, as shown by further increase in cell viability.

### REFERENCES

Aviram J, Samuelly-Leichtag G. Efficacy of cannabis-based medicines for pain management: A systematic review and meta-analysis of randomized controlled trials. Pain Physician, 2017, 20(6), E755-E796

Bisogno T, Hanus L, De Petrocellis L, Tchilibon S, Ponde DE, Brandi I, Moriello AS, Davis JB, Mechoulam R, Di Marzo V. Molecular targets for cannabidiol and its synthetic analogues: effect on vanilloid VR1 receptors and on the cellular uptake and enzymatic hydrolysis of anandamide. Br J Pharmacol, 2001, 134(4), 845-852

Burstein S. Cannabidiol (CBD) and its analogs: a review of their effects on inflammation. Bioorg Med Chem, 2015, 23(7), 1377-1385

Castex-Rizzi N, Galliano M F, Aries M F, Hernandez-Pigeon H, Vaissiere C, Delga H, Caruana A, Carrasco C, Lévêque M, Duplan H, Bessou-Touya S. In vitro approaches to pharmacological screening in the field of atopic dermatitis. Br J Dermatol, 2014, 170 (Suppl.), 12-18

Cerrato S, Brazis P, Della Valle M F, Miolo A, Puigdemont A. Inhibitory effect of topical adelmidrol on antigen-induced skin wheal and mast cell behavior in a canine model of allergic dermatitis. BMC Vet Res, 2012, 8, 230

Colombo I, Sangiovanni E, Maggio R, Mattozzi C, Zava S, Corbett Y, Fumagalli M, Carlino C, Corsetto P A, Scaccabarozzi D, Calvieri S, Gismondi A, Taramelli D, Dell'Agli M. HaCaT cells as a reliable in vitro differentiation model to dissect the inflammatory/repair response of human keratinocytes. Mediators Inflamm 2017, 7435621

Gupta D, Bleakley B, Gupta RK. Dragon's blood: Botany, chemistry and therapeutic uses. J Ethnopharmacol, 2008, 115(3), 361-380

Gupta D, Gupta RK. Bioprotective properties of Dragon's blood resin: In vitro evaluation of antioxidant activity and antimicrobial activity. BMC Complement Altern Med, 2011, 11(13)

Keppel J M, Kopsky D J. Treatment of chronic regional pain syndrome type 1 with palmitoylethanolamide and topical ketamine cream: modulation of nonneuronal cells. J Pain Res, 2013, 6, 239-245

Lever A R, Park H, Mulhern T J, Jackson G R, Comolli J C, Borenstein J T, Hayden P J, Prantil-Baun R. Comprehensive evaluation of poly(I:C) induced inflammatory response in an airway epithelial model. Physiol Rep, 2015, 3(4), e12334

Marini I, Bartolucci M L, Bortolotti F, Gatto MR, Bonetti GA. Palmitoylethanolamide versus a nonsteroidal anti-inflammatory drug in the treatment of temporomandibular joint inflammatory pain. J Orofac Pain, 2012, 26(2), 99-104

Muller C, Morales P, Reggio P. Cannabinoid ligands targeting TRP channels. Front Mol Neurosci, 2019, 11, 487

Namjoyan F, Kiashi F, Moosavi ZB, Saffari F, Makhmalzadeh BS. Efficacy of Dragon's blood cream on wound healing: A randomized, double-blind, placebo-controlled clinical trial. J Tradit Complement Med, 2016, 6(1), 37-40

Palmieri B, Laurino C, Vadalà M. A therapeutic effect of CBD-enriched ointment in inflammatory skin diseases and cutaneous scars. Clin Ther, 2019, 170(2), e93-e99

Peritore A F, Siracusa R, Crupi R, Cuzzocrea S. Therapeutic efficacy of palmitoylethanolamide and its new formulations in synergy with different antioxidant molecules present in diets. Nutrients, 2019, 11(9), E2175

Petrosino S, Cristino L, Karsak M, Gaffal E, Ueda N, Tilting T, Bisogno T, De Filippis D, D′Amico A, Saturnino C, Orlando P, Zimmer A, luvone T, Di Marzo V. Protective role of palmitoylethanolamide in contact allergic dermatitis. Allergy, 2010, 65(6), 698-711

Petrosino S, Di Marzo V. The pharmacology of palmitoylethanolamide and first data on the therapeutic efficacy of some of its new formulations. Br J Pharmacol, 2017, 174(11), 1349-1365

Petrosino S, Verde R, Vaia M, Allarà M, luvone T, Di Marzo V. Anti-inflammatory properties of cannabidiol, a nonpsychotropic cannabinoid, in experimental allergic contact dermatitis. J Pharmacol Exp, 2018, 365(3), 652-663

Pieters L, De Bruyne T, Van Poel B, Vingerhoets R, Totté J, Vanden Berghe D, Vlietinck A. In vivo wound healing activity of Dragon's Blood (Croton spp.), a traditional South American drug, and its constituents. Phytomedicine, 1995, 2(1), 17-22

Pona A, Cline A, Kolli S S, Taylor S L, Feldman S R. (2019) Review of future insights of Dragon's Blood in dermatology. Dermatol Ther, 2019, 32(2), e12786

Risco E, Ghia F, Vila R, Iglesias J, Alvarez E, Cañigueral S. Immunomodulatory activity and chemical characterisation of sangre de drago (dragon's blood) from Croton lechleri. Planta Med, 2003, 69(9), 785-794

Salatino A, Faria Salatino M L, Negri G. Traditional uses, chemistry and pharmacology of Croton species (Euphorbiaceae). J Braz Chem Soc, 2007, 18(1), 11-33

Sangiovanni E, Fumagalli M, Pacchetti B, Piazza S, Magnavacca A, Khalilpour S, Melzi G, Martinelli G, Dell'Agli M. Cannabis sativa L. extract and cannabidiol inhibit in vitro mediators of skin inflammation and wound injury. Phytother Res, 2019, 33(8), 2083-2093

Sheriff T, Lin M J, Dubin D, Khorasani H. The potential role of cannabinoids in dermatology. J Dermatolog Treat, 2019, 10, 1-7

Tóth K F, Ádam D, Bíró T, Oláh A. Cannabinoid signaling in the skin: therapeutic potential of the “c(ut)annabinoid” system. Molecules, 2019, 24(5), E918

Vaisberg A J, Milla M, Planas M C, Cordova J L, de Agusti E R, Ferreyra R, Mustiga M C, Carlin L, Hammond G B. Taspine is the cicatrizant principle in Sangre de Grado extracted from Croton lechleri. Planta Med, 1989, 55(2), 140-143

1. A composition comprising, as active agents, a combination consisting of (i) a cannabinoid or a Cannabis plant extract comprising said cannabinoid; (ii) Dragon's blood; and (iii) palmitoylethanolamide (PEA).

2. The composition of claim 1, wherein said cannabinoid is selected from the group consisting of cannabidiol (CBD), cannabidiolic acid (CBDA), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-THCVA, Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, or pharmaceutically acceptable salt thereof, Of and a mixture thereof

3. The composition of claim 2, wherein said cannabinoid is CBD, or an enantiomer, diastereomer, or a mixture thereof

4-5. (canceled)

6. The composition of claim 3, wherein the ratio between said cannabinoid and said Dragon's blood is from about 1:0.15 to about 1:30 by weight, respectively; the ratio between said cannabinoid and said PEA is from about 1:0.2 to about 1:20 by weight, respectively; and the ratio between said PEA and said Dragon's blood is from about 1:0.1 to about 1:20 by weight, respectively.

7. The composition of claim 6, wherein the concentration of said CBD or enantiomer, diastereomer, or mixture thereof in said composition from about 3 mg/mL to about 30 mg/mL; the concentration of said Dragon's blood in said composition is from about 5 mg/mL to about 100 mg/mL; and the concentration of said PEA in said composition from about 5 mg/mL to about 50 mg/mL.

8-15. (canceled)

16. The composition of claim 1, further comprising an agent selected from the group consisting of an antioxidant, a botanical extract, an oil, an odorant, a preservative, a thickening agent, an emollient agent, an emulsifying agent, a chelating agent, a moisturizing agent, a penetrating agent, and a cooling agent.

17. The composition of claim 16, wherein said antioxidant is vitamin C, vitamin E, alpha lipoic acid, vitamin A/retinol, co-enzyme Q10, or rosemary extract; said botanical extract is a cucumber extract, green tea extract, chamomile extract, bamboo extract, or aloe vera extract; said oil is hemp oil, neem oil, argan oil, manila oil, rose-hip oil, helichrysum oil, or grape seed oil; said odorant is an essential oil, or a natural fragrance; said preservative is phenoxyethanol, Liquid Germal Plus, Naticide, Leucidal Liquid, potassium sorbate, or benzoic acid;
said thickening agent is xantham gum, cellulose gum, Cera Bellina, or Emulsigel; said emollient agent is a triglyceride, lanolin, lecithin, or shea butter; said emulsifying agent is glyceryl stearate, Olivem 1000, a polysorbate such as polysorbate 20 and posysorbate polysorbate 80, BTMS, or Polawax; said chelating agent is tetrasodium EDTA; said moisturizing agent is glycerin, allantoin, panthenol, or isopropyl myristate; said penetrating agent is propylene glycol, mannitol, liposomes, sodium PCA, or squalene; and said cooling agent is menthol, WS-3, and Frescolat ML.

18. A pharmaceutical or cosmetic composition according to claim 1, for topical application to the skin or a mucous membrane of a subject.

19. The pharmaceutical or cosmetic composition of claim 18, formulated as a solution, emulsion, suspension, lotion, gel, cream, foam, ointment, paste, or patch.

22.-22. (canceled)

23. A method for treatment of an inflammatory disease or condition of the skin; treatment of a painful symptom of the skin, mucous membrane, or a superficial muscle; for minimizing scar formation after injury and pregnancy; for wound healing; or for anti-aging remedy, in a subject in need thereof, comprising topically applying to the skin or said mucous membrane of said subject an effective amount of a composition of claim 1.

24. (canceled)

25. The method of claim 23, wherein said inflammatory disease or condition of the skin is selected from the group consisting of psoriasis, acne, atopic and contact dermatitis, piles, itching, diaper rash, nipple cracks, eczema, and burns.

26. The method of claim 23, wherein said painful symptom of the skin, mucous membrane, or superficial muscle is spasm of the vaginal muscles, or painful piles burns.
